SEQUENCE LISTING (39 sequences)

SEQ ID NO: 1 - protein, 819 aa, Homo sapiens
MGSGARFPSGTLRVRWLLLLGLVGPVLGAARPGFQQTSHLSSYEIITPWRLTRERREAPRPYSKQVSYVIQAEGKEHIIHLERNKDLLPEDFVVYTYNKEGTLITDHPNIQNHCHYRGYVEGVHNSSIALSDCFGLRGLLHLENASYGIEPLQNSSHFEHIIYRMDDVYKEPLKCGVSNKDIEKETAKDEEEEPPSMTQLLRRRRAVLPQTRYVELFIVVDKERYDMMGRNQTAVREEMILLANYLDSMYIMLNIRIVLVGLEIWTNGNLINIVGGAGDVLGNFVQWREKFLITRRRHDSAQLVLKKGFGGTAGMAFVGTVCSRSHAGGINVFGQITVETFASIVAHELGHNLGMNHDDGRDCSCGAKSCIMNSGASGSRNFSSCSAEDFEKLTLNKGGNCLLNIPKPDEAYSAPSCGNKLVDAGEECDCGTPKECELDPCCEGSTCKLKSFAECAYGDCCKDCRFLPGGTLCRGKTSECDVPEYCNGSSQFCQPDVFIQNGYPCQNNKAYCYNGMCQYYDAQCQVIFGSKAKAAPKDCFIEVNSKGDRFGNCGFSGNEYKKCATGNALCGKLQCENVQEIPVFGIVPAIIQTPSRGTKCWGVDFQLGSDVPDPGMVNEGTKCGAGKICRNFQCVDASVLNYDCDVQKKCHGHGVCNSNKNCHCENGWAPPNCETKGYGGSVDSGPTYNEMNTALRDGLLVFFFLIVPLIVCAIFIFIKRDQLWRSYIRKKRSQTYESDGKNQANPSRQPGSVPRHVSPVTPPREVPIYANRFAVPTYAAKQPQQFPSRPPPPQPKVSSQGNLIPARPAPAPPLYSSLT

SEQ ID NO: 2 - protein, 29 aa, Homo sapiens
FNQHLCGSHLVEALYLVCGERGFFYTPKA

SEQ ID NO: 3 - protein, 21 aa, Homo sapiens
SQVSKNLESHHGDQMAQKSQS

SEQ ID NO: 4 - protein, 14 aa, Homo sapiens
LRAEQQRLKSQDLE

SEQ ID NO: 5 - DNA, 20 bp, Homo sapiens
cccctaggccctattcaaaa

SEQ ID NO: 6 - DNA, 20 bp, Homo sapiens
tgaactccctccacatagcc

SEQ ID NO: 7 - DNA, 20 bp, Homo sapiens
ctctgtgtggtgtcccagtg

SEQ ID NO: 8 - DNA, 20 bp, Homo sapiens
gggagtgagtgctccatcat

SEQ ID NO: 9 - DNA, 21 bp, Homo sapiens
aacaggaacttggaacaagca

SEQ ID NO: 10 - DNA, 20 bp, Homo sapiens
accaactgggacgacatgga

SEQ ID NO: 11 - DNA, 20 bp, Homo sapiens
gccatctcctgctcgaagtc

SEQ ID NO: 12 - DNA, 20 bp, Homo sapiens
tcaagaggcgaacacacaac

SEQ ID NO: 13 - DNA, 20 bp, Homo sapiens
tttccgcaacaagtcctctt

SEQ ID NO: 14 - DNA, 20 bp, Homo sapiens
ttcaggaagctctggaggaa

SEQ ID NO: 15 - DNA, 20 bp, Homo sapiens
ttctcctggtgtgcactctg

SEQ ID NO: 16 - DNA, 20 bp, Homo sapiens
tgcagtgacaggaacagtcc

SEQ ID NO: 17 - DNA, 20 bp, Homo sapiens
ggatgcatttcccatcctta

SEQ ID NO: 18 - DNA, 20 bp, Homo sapiens
caatgcccttcgacctaaaa

SEQ ID NO: 19 - DNA, 20 bp, Homo sapiens
ccacttcctttgcacactcg

SEQ ID NO: 20 - DNA, 19 bp, Homo sapiens
ctggctacagcaatatggc

SEQ ID NO: 21 - DNA, 21 bp, Homo sapiens
accttgggggtgtaattctgg

SEQ ID NO: 22 - DNA, 21 bp, Homo sapiens
gaagacgcggaggtccatgcc

SEQ ID NO: 23 - DNA, 21 bp, Homo sapiens
gagggcgtagcgcttccgacg

SEQ ID NO: 24 - DNA, 20 bp, Homo sapiens
ggggcttgcctcctagtatc

SEQ ID NO: 25 - DNA, 20 bp, Homo sapiens
ataggttttcccgacgtcct

SEQ ID NO: 26 - DNA, 20 bp, Homo sapiens
tggccaggagtactggaaag

SEQ ID NO: 27 - DNA, 20 bp, Homo sapiens
cagacctcgtaaccgtcctc

SEQ ID NO: 28 - DNA, 20 bp, Homo sapiens
catcctgtggtgtccatgag

SEQ ID NO: 29 - DNA, 20 bp, Homo sapiens
cagaagcaggggtcctgtag

SEQ ID NO: 30 - DNA, 20 bp, Homo sapiens
ccagatatcacccctgagga

SEQ ID NO: 31 - DNA, 20 bp, Homo sapiens
cttggggatagaagggttcc

SEQ ID NO: 32 - DNA, 20 bp, Homo sapiens
gatgtggagtgcctgatgtg

SEQ ID NO: 33 - DNA, 20 bp, Homo sapiens
tgcttgaccctcagagacct

SEQ ID NO: 34 - DNA, 20 bp, Homo sapiens
ccaagacggtcgttttgtct

SEQ ID NO: 35 - DNA, 20 bp, Homo sapiens
gttgaagcgccagtacctgt

SEQ ID NO: 36 - DNA, 20 bp, Homo sapiens
cagatgtggagtgccagatg

SEQ ID NO: 37 - DNA, 20 bp, Homo sapiens
tgtcagcagttccccataca

SEQ ID NO: 38 - DNA, 20 bp, Homo sapiens
agttcccggagtgagttgaa

SEQ ID NO: 39 - DNA, 20 bp, Homo sapiens
ctccactcctccctttcctc

(19) United States
(12) Patent Application Publication
(10) Pub. No.: US 2003/0091568 A1
(43) Pub. Date: May 15, 2003

(54) Inhibitors for the formation of soluble human CD23

(76) Inventor: Jurgen Frey, Bielefeld (DE)

Correspondence Address: FROMMER LAWRENCE & HAUG, 745 FIFTH AVENUE- 10TH FL., NEW YORK, NY, 10151, US

(21) Appl. No.: 10/238,011

(22) Filed: Sep. 9, 2002

Related U.S. Application Data
(63) Continuation-in-part of application No. 09/827,406, filed on Apr. 5, 2001, now abandoned.

(30) Foreign Application Priority Data
Apr. 7, 2000 (EP) 00 107 515.9

(51) Int. Cl. A61K 39/395; C07K 16/40

(52) U.S. Cl. 424/146.1; 530/388.26

(57) ABSTRACT

A pharmaceutical composition for the treatment or prophylaxis of disorders is described in which the overproduction of s-CD23 is implicated. This composition comprises an inhibitor for the formation of human soluble CD23, which inhibitor decreases or blocks selectively the activity of the metalloprotease ADAM9, which otherwise mediates the shedding of s-CD23 in human B cells and monocytes/macrophages. Also described is a pharmaceutical composition wherein the inhibitor for the formation of human soluble CD23 is a monoclonal or polyclonal antibody directed against the metalloprotease ADAM9. Such a pharmaceutical composition may be used in a method for selectively inhibiting the formation of s-CD23. It is a suitable medicament against inflammatory disorders, autoimmune diseases and allergy.

### RELATED APPLICATIONS

This application claims the benefit of and priority to European Patent Application 00 107 515.9, filed Apr. 7, 2000 and is a continuation-in-part application of U.S. application Ser. No. 09/827,406, filed Apr. 5, 2001. Each of the foregoing applications, as well as all documents cited or referenced in the foregoing applications. Also, all documents cited in this application and all documents cited or referenced in the herein cited documents are incorporated by reference.

### GOVERNMENT SUPPORT

Not Applicable.

### FIELD OF THE INVENTION

This invention relates to inhibitors for the formation of soluble human CD23 and for the treatment of conditions associated with excess production of soluble CD23 (s-CD23), such as allergy.

### BACKGROUND OF THE INVENTION

Matrix metalloproteinases (MMPS) such as collagenase, stromelysin and gelatinase are involved in connective tissue breakdown. Classes of matrix metalloproteinase inhibitors include derivatives of hydroxamic acid, barbituric acid, phosphonates and thiols.

International patent application WO 93/20047 discloses that inhibitors of the matrix metalloproteinases, especially derivatives of hydroxamic acid, can be useful for the treatment of prophylaxis of conditions involving tissue breakdown, for example rheumatoid arthritis (RA), osteopenias such as osteoporosis, periodontitis, gingivitis, corneal epidermal or gastric ulceration, and tumor metastasis or invasion.

The low affinity receptor for IgE, FcεRII (CD23), is a type two membrane glycoprotein belonging to the C-type (calcium dependent) lectin family (Kikutani et al., 1986). Some members of this family have been shown to be adhesion molecules. The lectin domain of CD23 comprises the IgE binding site (Bettler et al., 1989). IgE binding is a calcium dependent process. CD23 is expressed on a variety of hematopoietic cell types such as B and T lymphocytes, a subset of thymic epithelial cells, monocytes, macrophages, follicular dendritic cells (FDC), platelets, eosinophils, Epstein-Barr virus (EBV)-containing nasopharyngeal carcinoma cells, Langerhans cells and natural killer (NK) cells (Delespesse et al., 1991).

Human CD23⁺ cells can express two different forms of CD23, CD23a and CD23b (Yokota et al., 1988), which differ only at the N-terminal cytoplasmic region (7 amino acids), whereas the C-terminal extracellular region is identical. Both forms are derived from one gene by utilizing different transcriptional initiation sites and alternative RNA splicing. CD23a is constitutively expressed only on mature B cells (IgM⁺/IgD⁺) in the periphery and B cell lines, whereas IgM⁺/IgD⁺ B cells in the bone marrow (BM) are CD23⁺. B cells in the follicular mantle of tonsils are CD23⁺, those in the germinal center are CD23⁺. The expression of CD23 can be strongly induced by interleukin 4 (IL-4), known to induce germline IgE transcription (Gordon et al., 1991), but is lost after isotype-switching to IgG, IgA and IgE.

In some malignant pre-B cells from acute lymphoblastic leukemia patients, CD23 expression can also be induced by IL-4. Signals delivered via CD40 on B cells (Clark, 1990) strongly potentiate the IL-4 induced induction of CD23 on mature B cells. This second signal is provided by physical interaction of B cells with T cells expressing CD40 ligand (CD40L). Furthermore, IL-13 and IL-4, both known to increase CD23 expression, also induce the production of IgE in normal B cells due to isotype switching (Punnonen et al., 1993; Lebman et al., 1988). Factors counteracting the IL-4 induced CD23 expression (INF-γ, INF-α and PGE2) also block IgE synthesis by B cells (Defrance et al., 1987).

CD23b is mainly found on activated monocytes, macrophages, eosinophils, dendritic cells, platelets, and transiently on IL-4 treated B cells (Munoz et al., 1998). Ligation of CD23 on human monocytes triggers monokine release. Monocyte activation can be regulated by the specific interaction of CD23 with the α chains of the β2 integrin adhesion molecule complexes CD11b-CD18 and CD11c-CD18, causing an increase in nitrogen oxide (NO) and oxidative product (H₂O₂), as well as proinflammatory cytokines (IL-1β, IL-6, and TNFα). Increased levels of CD23 are found in different chronic inflammatory diseases such as RA, SLE and glomerulonephritis. Consistent with these findings are results obtained for CD23 in collagen-induced arthritis (CIA) in mice, a model for RA. The percentage of CD23⁺ lymph node cells increased after collagen type II immunization. In contrast, CD23-deficient mice developed CIA with a delayed onset and reduced severity (Kleinau et al., 1999).

Transient induction of CD23 by IL-4 on the plasma membrane of B cells and monocytes is accompanied by concomitant proteolytic release (shedding) of different defined soluble CD23 (s-CD23) fragments (Letellier et al., 1990; and Letellier et al., 1989). Different cytokine activities have been attributed especially to the soluble 25 kDa form of CD23. s-CD23 has been shown to act as an autocrine growth factor in some EBV-transformed B cell lines and as a differentiation promoting factor for prothymocytes. Furthermore, s-CD23 can prevent apoptosis of germinal center B cells, most likely via induction of bcl-2 induction (Liu et al., 1991).

The most convincing evidence for s-CD23 as a key regulatory molecule in IgE response has been shown recently (Mayer et al., 2000). Inhibition of CD23 processing correlates with inhibition of IL-4 stimulated IgE production in human peripheral blood lymphocytes (PBL) and human peripheral blood lymphocytes (hu-PBL) reconstituted severe combined immuno deficiency (SCID) mice. Mayer et al. show that hu-PBL, reconstituted into SCID mice, demonstrate a significantly enhanced IgE response when challenged (immunized) with a synthetic allergen (dinitrophenyl-labelled ovalbumin) in the presence of the 33 kDa form of s-CD23.

The mechanisms by which s-CD23 fragments are generated have not been well characterized. Batimastat, as well as a number of other hydroxamic acid-based metalloproteinase inhibitors, inhibit proteolytic processing of CD23 in nanomolar concentrations (Christie et al., 1997; Wheeler et al., 1998; Bailey et al., 1998a; Bailey et al., 1998b). In a more recent attempt to characterize the proteinase involved in CD23 shedding, a CD23 processing activity was enriched by gel chromatography of human B cell line RPMI8866 plasma membrane fractions (Marolewski et al., 1998).

The ectodomain shedding of membrane proteins has common features. In most, proteolytic release can be blocked by hydroxamic acid-based inhibitors, processing occurs at a fixed distance from the plasma membrane, and shedding can be induced by activation of protein kinase C (PKC) (Hooper et al., 1997). The shedding of pro-TNF-α by ADAM17 (TACE) is the first example showing that a metalloproteinase is involved (Black et al., 1997; Moss et al. 1997). TACE is a member of the growing ADAM (A Disintegrin And Metalloproteinase) or MDC (Metalloproteinase Disintegrin Cystein-rich) family (for reviews see Black and White, 1998 and Schlöndorff and Blobel, 1999) all having a conserved metalloprotease domain, but only 15 (out of 28) are supposed to be active metalloproteases because of the highly conserved consensus sequence (HEXXH) which is part of the catalytic domain (Bode et al., 1993). (ADAM9 is a synonym for MDC9, originally used by Prof. Dr. Carl P. Blobel, see Weskamp et al., 1996). The currently used nomenclature for all originally named MDC proteins is ADAM proteins.

### SUMMARY OF THE INVENTION

It has now been found that the metalloproteinase ADAM9, which is widely expressed (Weskamp et al., 1996), is involved in shedding human CD23b. It has been demonstrated that only ADAM9, but not ADAM10 and ADAM17 (TACE), either overexpressed in COS cells and CHO cells or endogenously expressed in human B cells, leads to a significant reduction of surface bound CD23b coexpressed in these cells. Human B cell lines overexpressing ADAM9 show a marked reduction in surface CD23. In addition, it has been shown that upregulation of c-myc in human B cells leads to a significant increase in ADAM9 expression accompanied by increased shedding (40%) of CD23, i.e. production of s-CD23. Shedding of CD23 in transfected COS and CHO cells, as well as B cells endogenously expressing ADAM9 and CD23, can be blocked by the metalloproteinase inhibitor batimastat (BB-94), but not by tissue inhibitors of metalloproteinases (TIMP)-1, -2 and -3. These inhibitor studies therefore exclude all known human MMPs, membrane type matrix metalloproteinases (MT-MMPs) as well as ADAM10 and ADAM17 as being involved in CD23 shedding.

These results led to the first subject of the present invention, which is a composition for the treatment or prophylaxis of allergy in which the overproduction of s-CD23 is implicated, which comprises an inhibitor for the formation of human soluble CD23, wherein the inhibitor is a compound which selectively decreases or blocks the activity of the metalloproteinase ADAM9, which otherwise mediates the shedding of s-CD23 in human B cells as well as all other CD23 positive cells, all of which also express ADAM9.

A further subject of the invention is an antibody that selectively binds to the metalloproteinase ADAM9. Preferably this antibody is monoclonal and humanized.

A further subject of the invention is a composition, wherein the inhibitor for said ADAM protein is an antibody which is selectively directed against the metalloproteinase ADAM9, or is a synthetic inhibitor from the group of hydroxamic acid based or barbituric acid based inhibitors which selectively decreases or blocks the activity of the metalloproteinase ADAM9.

Further subjects of the invention are antibodies which selectively bind to the metalloproteinase ADAM9 and their use in the manufacture of medicaments which are useful for the treatment of patients suffering from allergy which is associated with an overproduction of s-CD23.

Finally, the invention is directed to the use of antibodies for the treatment of said patient.

### BRIEF DESCRIPTION OF THE DRAWINGS

FIG. 1 shows release of surface CD23 by overexpression of ADAMs in COS cells.

FIG. 2 shows release of surface CD23 in CHO cells expressing Tet-regulated ADAM9.

FIG. 3 shows that ADAM9 mediated CD23 release in CHO cells is not inhibited by Tissue Inhibitors of Metalloproteinases (TIMPs).

FIG. 4 shows eneration of s-CD23 fragments in transfected CHO cells.

FIG. 5 shows the influence of Tet-regulated c-myc on the expression of MMPs and ADAMs in P493-6 cells.

FIG. 6 shows that s-CD23 release from P493-6 cells (c-myc “On”) is not inhibited by TIMPs.

FIG. 7 shows the generation of soluble ADMA9-Fc.

FIG. 8 shows a mass spectrum of cleavage of Peptide #147.

FIG. 9 shows a mass spectrum of cleavage of Peptide #148.

### DETAILED DESCRIPTION OF THE INVENTION

The foregoing findings suggest that ADAM9 is involved in the formation of s-CD23 and that, by the inhibition or blocking of ADAM9, the amount of s-CD23 may be drastically reduced. This aim can be achieved on several ways.

One route for the inhibition of ADAM9 is based on antisense technology. Antisense technology is emerging as an effective means of lowering the levels of a specific gene product. It is based on the findings that these “antisense” sequences hybridize a gene or associated target polynucleotide, to form a stable duplex or triplex, based upon Watson-Crick or Hoogsteen binding, respectively. The specifically bound antisense compound then either renders the respective targets more susceptible to enzymatic degradation, blocks translation or processing, or otherwise blocks or inhibits the function of a target polynucleotide. Where the target polynucleotide is RNA, the antisense molecule hybridizes to specific RNA transcripts disrupting normal RNA processing, stability, and translation, thereby preventing expression of the targeted gene.

Administration of antisense oligonucleotides or transfer of expression constructs capable of producing intracellular antisense sequences complementary to the mRNA of interest have been shown to block the translation of specific genes in vitro and in vivo. For example, Holt et al. (1988), focusing on c-myc, found the formation of an intracellular duplex with target mRNA and a selective decrease of c-myc protein in human promyelocytic leukemia HL-60 cells.

According to the present invention the shedding of CD23 can not only be blocked by the inhibition of ADAM9 but also by any means suitable to inhibit c-myc. This can be achieved by an antisense oligonucleotide specific for c-myc. Therefore the present invention provides a method for inhibiting the generation of ADAM9 by administering to a patient suffering from overproduction of s-CD23 an antisense oligonucleotide complementary to a region of mRNA encoding c-myc. Such antisense oligonucleotide may be used in the manufacture of a medicament by which therapeutically effective amounts are administered to a patient needing a reduction of s-CD23 levels.

Another successful route for the inhibition or blocking of ADAM9 comprises the administration of a synthetic inhibitor from the group of hydroxamic acid based or barbituric acid based inhibitors.

A further successful route for the inhibition of ADAM9 consists in the production of monoclonal or polyclonal antibodies that are selectively directed against said metalloproteinase. Such antibodies have been generated in mice by known methods, and the murine antibodies have later been humanized.

Antibodies can be obtained by any method known in the art. They are most conveniently obtained from hybridoma cells engineered to express an antibody. Methods of making hybridomas are well known in the art. The hybridoma cells can be cultured in a suitable medium, and spent medium can be used as an antibody source. Polynucleotides encoding the antibody can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Another method of obtaining antibodies is to immunize suitable host animals with an antigen and to follow standard procedures for polyclonal or monoclonal production. Antibodies thus produced can be humanized by methods known in the art. Examples of humanized antibodies are provided, for instance, in U.S. Pat. Nos. 5,530,101 and 5,585,089.

“Humanized” antibodies are antibodies in which at least part of the sequence has been altered from its initial form to render it more like human immunoglobulins. In one version, the heavy chain and light chain C regions are replaced with human sequence. In another version, the complementarity-determining regions (CDR) comprise amino acid sequences for recognition of antigen of interest, while the variable framework regions have also been converted to human sequences. See, for example, EP 0329400. In a third version, variable regions are humanized by designing consensus sequences of human and mouse variable regions, and converting residues outside the CDR that are different between the consensus sequences. This invention encompasses humanized antibodies. The humanized antibodies have proven to be valuable and selective inhibitors of ADAM9, and can be used for the manufacture of a medicament for the treatment of disorders which are associated with overproduction of soluble human CD23 by administering a therapeutically effective amount of said antibody.

The term “antibody” as used in this invention includes intact immunoglobulin molecules as well as fragments thereof, such as Fab and Fab′, which are capable of binding the epitopic determinant. Fab fragments retain an entire light chain, as well as one-half of a heavy chain, with both chains covalently linked by the carboxy terminal disulfide bond. Fab fragments are monovalent with respect to the antigen-binding site.

The amino acid sequence of human ADAM9 is given by SEQ ID NO:1. As used herein, the term “amino acid sequence” is synonymous with the term “polypeptide” and/or the term “protein”. The amino acid sequence may be prepared/isolated from a suitable source, or it may be made synthetically or it may be prepared by use of recombinant DNA techniques.

The compositions of the invention can be administered in a pharmaceutically acceptable excipient, such as water, saline, aqueous dextrose, glycerol, or ethanol. The compositions can also contain other medicinal agents, pharmaceutical agents, adjuvants, carriers, and auxiliary substances such as wetting or emulsifying agents, and pH buffering agents.

Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

A “therapeutically effective amount” is an amount sufficient to effect a beneficial or desired clinical result. A therapeutically effective amount can be administered in one or more doses. In terms of treatment, an effective amount is an amount that is sufficient to palliate, ameliorate, stabilize, reverse or slow the progression of a disease or condition associated with excess production of sCD23. A therapeutically effective amount can be provided in one or a series of administrations. In terms of an adjuvant, an effective amount is one sufficient to enhance the immune response to the immunogen. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, the severity of the condition and the form of the composition being administered.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow release or sustained release forms, whereby a relatively consistent level of the active compound are provided over an extended period.

An “individual”, “patient” or “subject” is a vertebrate, preferably a mammal, most preferably a human. Mammals include, but are not limited to, humans, farm animals, sport animals, and pets.

For the purposes of this application, a molecule, compound or agent that selectively decreases or blocks the activity of metalloprotease ADAM9 can be identified by the assays described in the Examples, particularly in Example 9 under “Influence of metalloproteinase inhibitors on CD23 shedding”.

The following Examples are provided to illustrate the invention, and should not be considered to limit its scope in any way.

### EXAMPLE 1

Ectodomain Shedding of CD23 in COS Cells is Mediated by ADAM9 but not by ADAM10 and ADAM 17

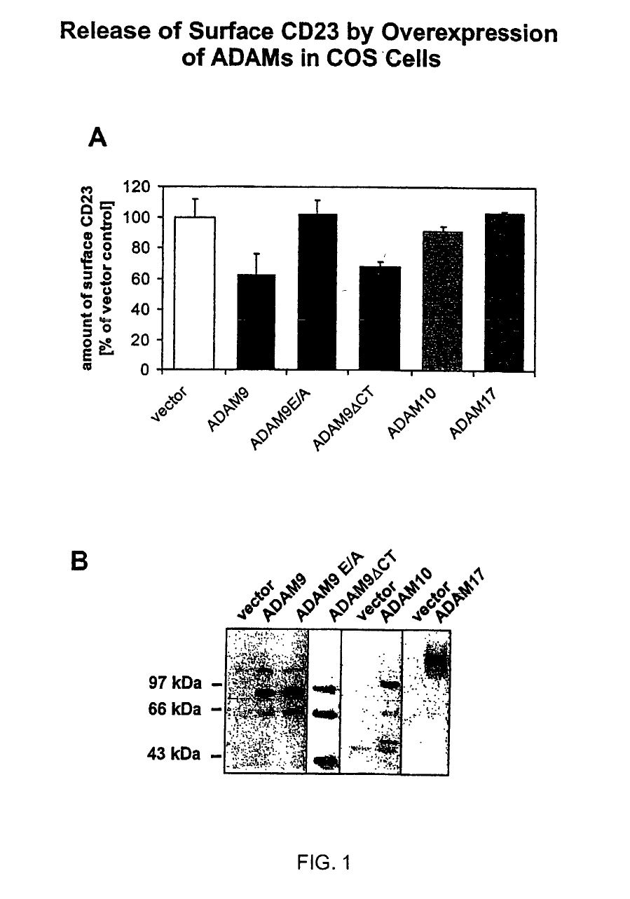

Evidence that ADAM9 is a sheddase for membrane bound CD23 comes from cotransfection experiments. COS cells were transfected with human CD23b cDNA, together with either ADAM9, ADAM9E/A mutant (catalytically inactive), ADAM9ACT (truncated cytoplasmic domain), ADAM10, or ADAM17 (TACE); ADAM10 and ADAM17 have been shown to also be expressed in human B cell lines. Flow cytometry analyses (FACS) 48 h after cotransfection show that only ADAM9 and ADAM9ACT expression, but not the catalytically inactive ADAM9E/A mutant, ADAM10 or ADAM17 (TACE), led to the proteolytical release of CD23, as measured by the decrease in surface fluorescence intensity by 35-40% of the vector control (FIG. 1A). The synthesis of all transfected ADAM cDNAs was verified by Western blot analyses using specific antibodies (FIG. 1B). Thus, ectopic overexpression of ADAM9 results in the ectodomain processing of CD23 thus generating soluble CD23 (s-CD23).

### EXAMPLE 2

Tetracyclin-Mediated Upregulation of ADAM9 in CHO Cells Leads to the Generation of s-CD23

To exclude the possibility that transient overexpression of ADAM9 in COS cells leads to the release of other metalloproteinases which might be responsible for the observed shedding, stable CHO (Chinese Hamster Ovary) cell clones were generated which constitutively express CD23 and allow inducible ADAM9 transcription by withdrawal of tetracycline (TC) from the medium (CHO M8 cells).

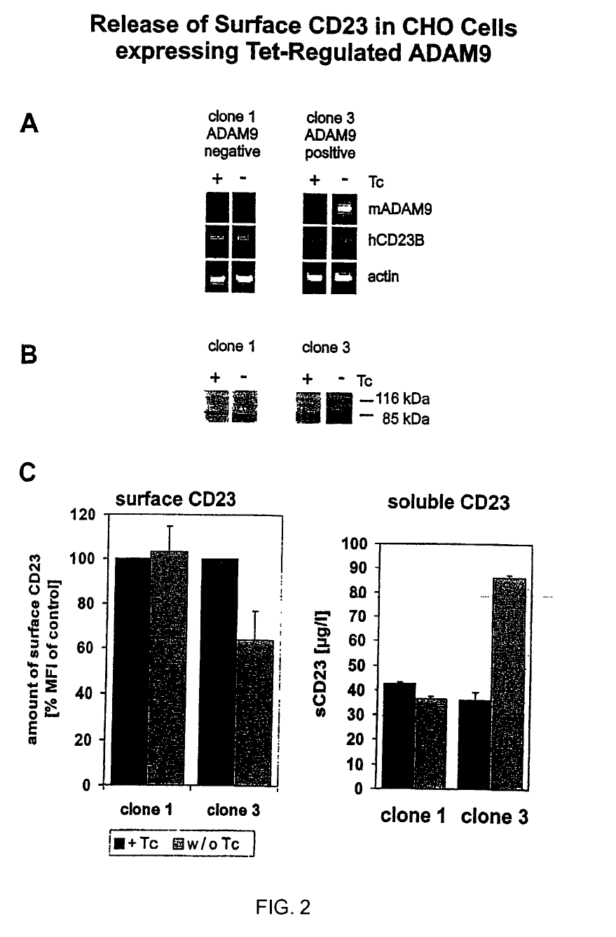

CHO AA8 cells (Tet-Off cells, Clontech) were cotransfected with CD23B under the control of the SV40 promotor, together with ADAM9, under the control of the CMV minimal (CMVₘᵢₙ) promoter. Clones were selected for constitutive CD23 and tet-regulatable ADAM9 expression. In clone 1, the ADAM9 cDNA construct is integrated into a locus where it is kept silent, whereas CD23b is constitutively expressed (FIG. 2A: RT-PCR analyses, 30 PCR cycles). In contrast, CD23 as well as actin mRNA transcripts could be easily detected after 20 PCR cycles. In Western blot analyses of whole cell extracts using an antibody raised against the disintegrin domain of ADAM9 (anti-AD9Dis), only faint bands of Mᵣ 85 kDa could be detected in cells of clone 1 (FIG. 2B), which could be due to cross-reaction with endogenous hamster ADAM9. This clone was used as ADAM9 negative control. Clone 3, showing a strong tet-regulatable ADAM9 expression, as well as a constitutive CD23b expression, was used for all studies. The observed induction in ADAM9 mRNA (analyzed by RT-PCR) led to a dramatic increase in ADAM9 protein synthesis, as judged by Western blotting using anti-AD9Dis antibodies. Besides the mature 85 kDa ADAM9 form, some unprocessed protein with a Mᵣ of 116 kDa could also be detected (FIG. 2B).

Removal of tetracycline from the culture medium of clone 3, leading to an upregulation of ADAM9, caused a 40-50% decrease in surface bound CD23 as measured by flow cytometry (MFI: mean fluorescence intensity), whereas the number of surface CD23 molecules in clone 1 was not changed under these conditions (FIG. 2C). The reduction in the number of surface molecules correlates with the increase in soluble CD23 (s-CD23), determined by ELISA (The Binding Site ELISA kit; FIG. 2C).

### EXAMPLE 3

Exclusion of the Involvement of Metalloproteinase (MMP) Activities Other than ADAM9 in CD23 Shedding

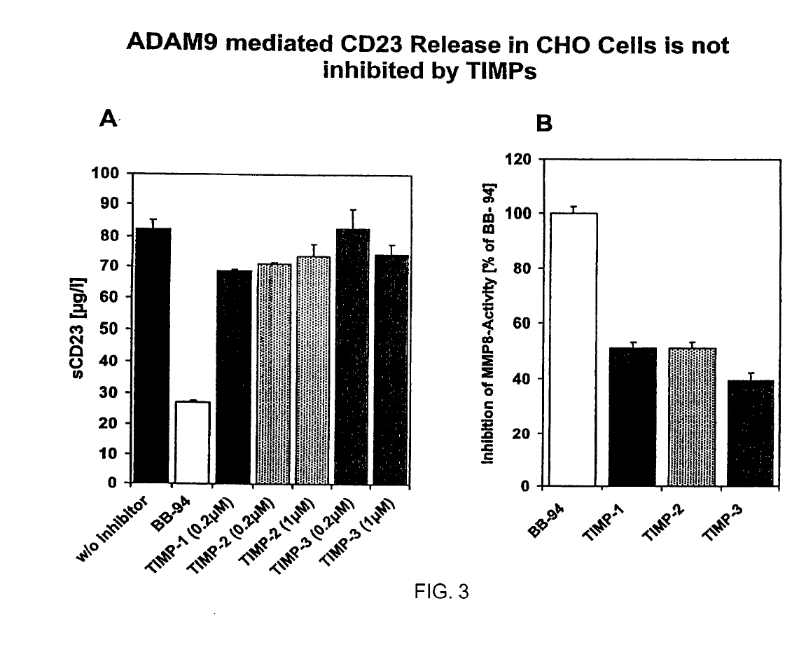

CHO AA8 cells (Clontech), which constitutively express human CD23b and a tet regulatable ADAM9 (clone 3, described above), were cultured in the absence of tetracycline (ADAM9 expression “On”) as well as the presence or absence of the metalloproteinase inhibitor batimastat (BB-94, British Biotechnology) as a broad spectrum MMP inhibitor, as well as various concentrations of either TIMP-1, TIMP-2 or TIMP-3. After 16 hours, aliquots of the respective culture supernatants were analysed by ELISA for the formation of s-CD23 (The Binding Site). In contrast to batimastat, none of the TIMPs studied could block the proteolytic release of CD23 from the membrane (FIG. 3A). The concentrations of the various TIMPs used in these studies have been shown to block the enzymatic activities of all known secreted MMPs, membrane-type matrix metalloproteinases (MT-MMPs) as well as ADAM10 and ADAM17. Because shedding of CD23 could not be inhibited by TIMP-1, -2 and -3 (see FIG. 3), one can exclude metalloproteinase activities other than ADAM9 as responsible for the proteolytic release of CD23 from the membrane. To exclude the possibility that the TIMPs or BB-94 either became inactive during the incubation period or were rapidly endocytosed by the CHO cells, aliquots of the culture supernatants (after 16 h) were analyzed for inhibition of MMP8 activity. These studies show that all inhibitors used were still present and biologically active in the culture supernatant after a 16 h incubation (FIG. 3B).

From these studies one can therefore exclude that endogenous CHO cell metalloproteinase activities are responsible for the CD23 release. Furthermore, these studies also show that ADAM9 is the responsible sheddase under the experimental conditions used, as shedding cannot be inhibited by either TIMP-1, -2, -3.

### EXAMPLE 4

ADAM9-Mediated Shedding of CD23 Generates a Soluble 33 kDa Fragment of CD23

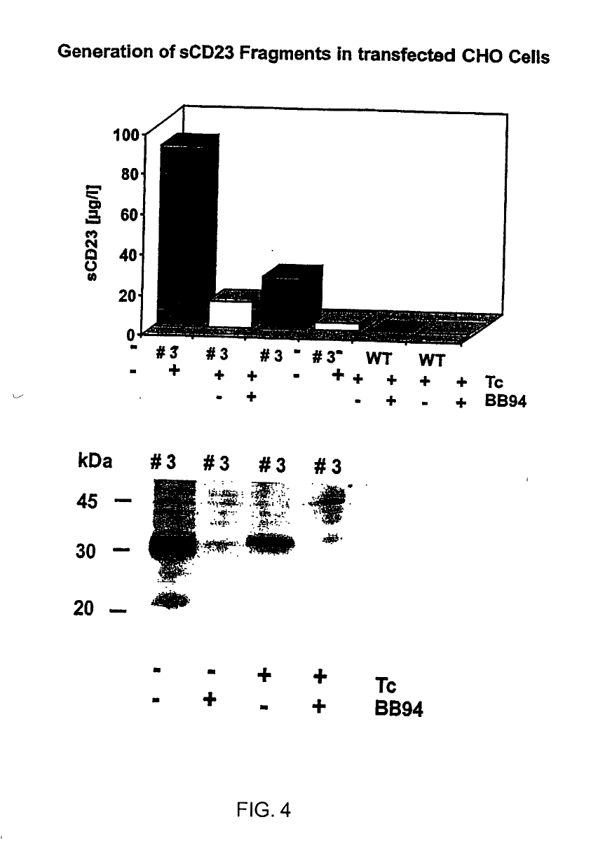

To prove whether the proteolytic release of CD23 from CHO AA8 cells upon upregulation of ADAM9 expression (see Example 3) leads to the generation of a biologically active form of s-CD23, s-CD23 proteins from culture supernatants were analyzed by Western blot analyses. CHO M8 clone 3 cells were cultured in the presence (ADAM9 expression “Off”) or absence (ADAM9 expression “On”) of tetracylin (Tc). Under both conditions, the influence of batimastat (BB-94) on CD23 shedding was analyzed. The concentration of s-CD23 in the culture supernatants was determined by ELISA. In parallel, the produced s-CD23 cleavage products were analyzed by Western blot analyses (FIG. 4). Untransfected CHO AA8 cells (WT) were used as a negative control in the ELISA assays. In the absence of Tc (ADAM9 expression “On”), about 2-3 times the amount of s-CD23 was produced compared to cells kept in the presence of Tc (ADAM9 expression “Off”). This proteolytic relase could be almost blocked by the addition of BB-94. The 33 kDa form of s-CD23 and, in minor amounts, the 25 kDa cleavage fragment were detected by Western blot analyses. The fragment size is identical to that observed by different laboratories after culturing the human B cell line RPMI 8866 in the absence of serum (spontaneous release of CD23) (Marolewski et al., 1998). The 33 kDa form of s-CD23 has recently been shown to significantly enhance the IgE response of human peripheral blood lymphocytes using a SCID mouse model. Inhibition of CD23 processing correlates with inhibition of IL-4-stimulated IgE production in human PBL and hu-PBL-reconstituted SCID mice (Mayer et al, 2000). Thus, overexpression of ADAM9 in transfected CHO cells leads to the production of the biologically active soluble 33 kDa form of CD23 shown to potentiate the IgE response to allergens.

### EXAMPLE 5

Expression of MMPs, MT-MMPs and ADAMs in the Human B-Cell Line P493-6 Containing a Tet-Regulatable c-myc Gene

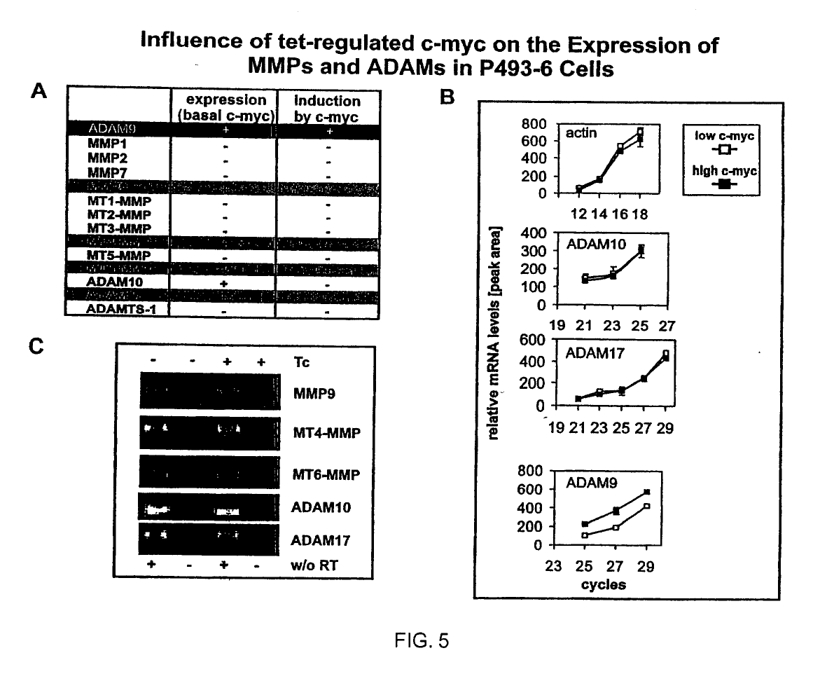

To study the effect of endogenous ADAM9 on endogenously expressed CD23, the human B cell line P493-6 was used. This cell line is derived from the conditionally EBV-transformed parental cell line EREB2-5 by transfection with a tetracycline regulatable c-myc (Tet “Off”) (Kempkes et al., 1995; Schuhmacher et al., 1999). The influence of basal and enhanced c-myc expression on the expression of CD23, actin, MMPs, MT-MMPs, as well as ADAMs known or predicted to be catalytically active (including ADAMTs1), were analyzed by RT-PCR using specific primers (FIG. 5A). Among the ADAMs known or predicted to be catalytically active based on their conserved amino acid (aa) sequence motif, HEXXH, within the catalytic domain, P493-6 cells only express ADAM9, ADAM10 and ADAM17. mRNA levels of the MMPs, MT-MMPs and ADAMs were quantified by laser densitometry of the RT-PCR products separated by agarose gel electrophoresis (examples shown in FIG. 5C). The amounts of RT-PCR products were analyzed after different numbers of PCR cycles to allow analyses in the linear range (FIG. 5B). Among the transcripts analyzed by RT-PCR, the level of c-myc RNA increased about 4 fold upon withdrawal of tetracycline (TC). Interestingly, the level of ADAM9 mRNA increased by about 30% after upregulation of c-myc (FIG. 5B). c-myc upregulation also led to a 60-70% increase in CD23a transcript which is constitutively expressed in B cells whereas the level of CD23b was not affected. In contrast, all other MMPs, MT-MMPs as well as ADAM10, ADAM17 and ADAMTs1 mRNA levels remained constant. Thus, this human B cell line expressing endogenous CD23 allows the study of the effect of enhanced ADAM9 expression on CD23 shedding in a similar manner to the tet-regulatable ADAM9 CHO M8 cell system described in Example 2 (see FIG. 2).

### EXAMPLE 6

Shedding of CD23 from B Cells (P493-6) is not Inhibited by TIMP-1, TIMP-2 and TIMP-3

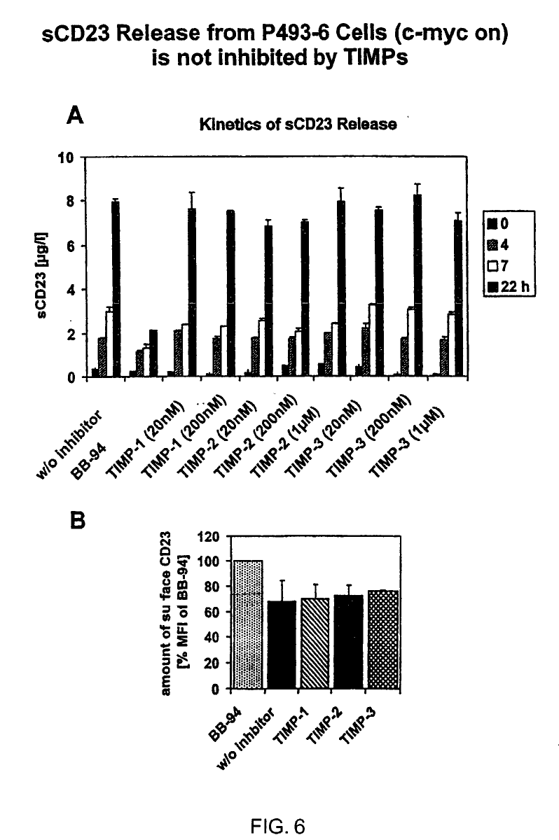

TIMP-1, -2 and -3 are known to inhibit all known secreted matrix metalloproteinases (MMPs), membrane-type matrix metalloproteinases (MT-MMPs), as well as ADAM10 (inhibited by TIMP-1 and TIMP-3) and ADAM17 (TACE, inhibited by TIMP-3 only). In contrast, it has been shown that membrane bound ADAM9 is not inhibited by either TIMP-1, -2 or -3 (see Example 3, FIG. 3). Using the human B cell line P493-6 with a tetracycline regulatable c-myc gene allowed the upregulation of endogeneous ADAM9 via upregulation of c-myc. Under these conditions increased shedding of endogeneous membrane bound CD23, as analyzed by the decrease in surface number (analyzed by FACS analysis), which could be blocked by batimastat (BB-94) but not by TIMP-1, TIMP-2 or TIMP-3 (FIG. 6B), was observed. The decrease in cell surface bound CD23 correlated with the increase in the s-CD23 concentration in the culture medium assayed by ELISA (CD23 ELISA kit, Becton Dickinson, and CD23 ELISA kit, the Binding Site). Shedding of CD23 in P493-6 cells could not be inhibited by TIMP-1, -2 and -3 at concentrations known to inhibit MMPs, MT-MMPs, as well as ADAM10 and ADAM17 but not ADAM9 (FIG. 6a). Thus, the data obtained with human B cells endogenously expressing ADAM9 and CD23 are consistent with the results obtained with either transfected COS cells (FIG. 1) or transfected CHO cells (see FIG. 3). Therefore, all known MMPs, MT-MMPs, as well as ADAM10 and ADAM17 (TACE), which are also both expressed in P493-6 B cells, can be excluded as involved in the proteolytic release of CD23 in B cells. These studies clearly show that endogenous ADAM9 is responsible for the shedding of endogenously expressed CD23 in human B cells.

### EXAMPLE 7

Generation of Catalytically Active Soluble ADAM9-Fc

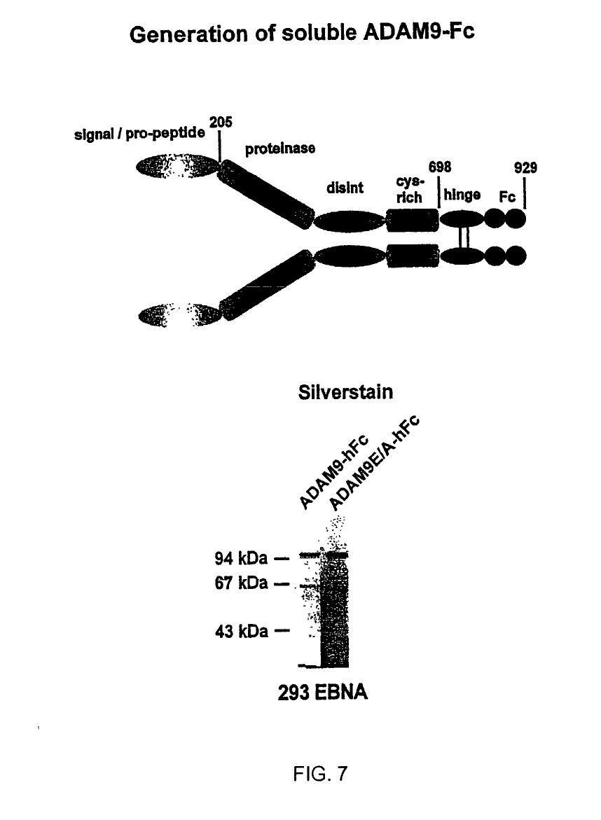

In order to study the substrate specificity of ADAM9 with synthetic CD23 peptides containing the putative cleavage sites, a soluble form of ADAM9 containing all ectodomains was generated. For this purpose, the complete extracellular domain of ADAM9 (aa 1-698) or the respective inactive mutant ADAM9E/A was fused in frame to the human IgG1 Fc cDNA fragment (FIG. 7). This chimeric cDNA construct (ADAM9 from aa 1-698; aa 699-929 of human or mouse IgG1 Fc,) was cloned into the eukaryotic expression vector pCEP4 (Invitrogen) and transfected into HEK293-EBNA1 cells. Stable cell clones producing the respective ADAM9-Fc fusion protein were adapted to grow under serum-free conditions. The recombinant fusion proteins were purified to homogeneity on Protein A Sepharose from serum-free culture supernatants. Both recombinant ADAM9-Fc fusion proteins were detected by Western blot analyses using either antibodies against the disintegrin domain of ADAM9 (anti-AD9Dis) or anti-human IgG1-Fc (FIG. 7). The observed Mᵣ of about 100 kDa corresponds with the expected mass of the mature (processed) form of ADAM9-Fc. The bands with Mᵣ 65-68 kDa are degradation products of the fusion proteins.

### EXAMPLE 8

Identification of the Cleavage Site in CD23 by Soluble ADAM9

According to the s-CD23 fragments generated in vivo (see FIG. 4, transfected CHO cells), synthetic CD23 peptides containing the putative cleavage site of the 37 kDa (peptide #148, containing aa Ser⁶⁵-Ser⁸⁵ of CD23) and 33 kDa (peptide #147, containing aa Leu⁹⁵-Glu¹⁰⁸ of CD23) fragments, respectively, were used and their cleavage by catalytically active, soluble ADAM9 was analyzed (see below).

Assay of the Catalytic Activity of Soluble ADAM9-Fc Fusion Protein

| | |
|---|---|
| a) | Cleavage of the oxidized human insulin B chain |
| | oxidized insulin B chain (MW: 3494,649 g/mol) |
| | ↓ ↓ |
| | FNQHLCGSHLVE ALY LVCGERGFFYTPKA (SEQ ID NO:2) |

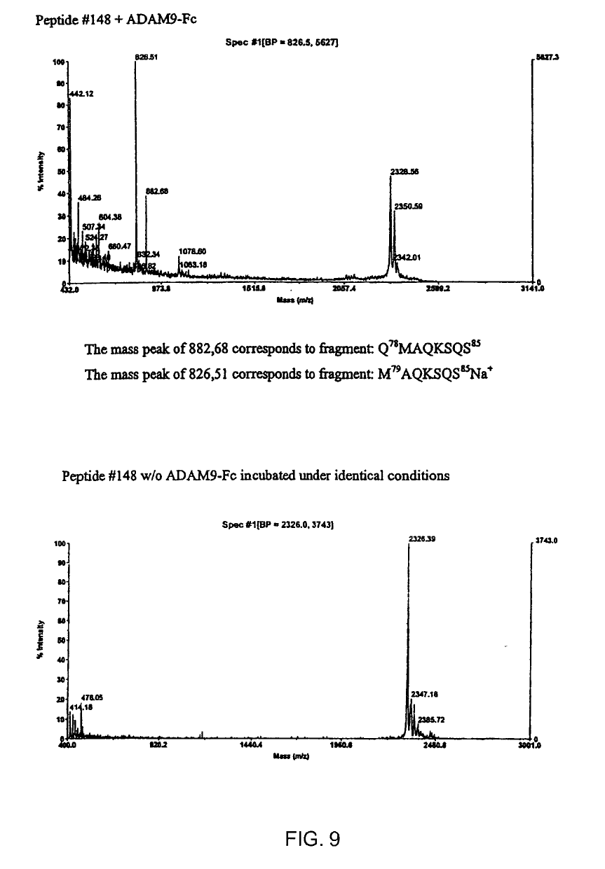

The estimated cleavage sites (marked by an ↓), determined by mass spectrometry (MALDI-TOF), are indicated. Cleavage of the insulin B chain was completely inhibited in the presence of 2 mM o-phenanthroline, known to block MMPs by chelating Zn²⁺ ions. In contrast to earlier findings using only the recombinant catalytic domain of ADAM9 (Roghani et al., 1999), a higher specificity was observed in that there were only two cleavage sites within the insulin B chain using all extracellular domains of the mature catalytically active ADAM9 (ADAM9-Fc fusion protein).

| | |
|---|---|
| b) | Cleavage of synthetic s-CD23 peptides con- |
| | taining the putative cleavage sites Peptide |
| | #148 contains the putative cleavage site of the |
| | 37 kDa s-CD23 cleavage product. (MW: 2325.70 |
| | g/mol; see FIG. 9, MALDI-TOF spectra) |
| | @ @ |

(SEQ ID NO:3)

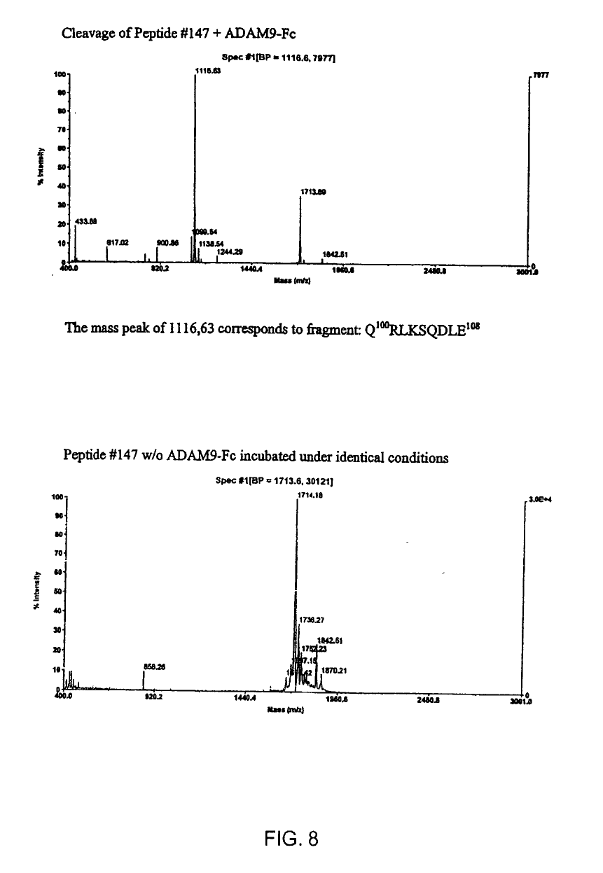

H-S⁶⁵QV SKN LES HHG D Q M AQK SQS⁸⁵-OH
Peptide #147 contains the putative cleavage
site of the 33 kDa s-CD23 cleavage product.
(MW: 1713.89 g/mol; see FIG. 8, MALDI-TOF
spectra)
@

(SEQ ID NO:4)

H-L⁹⁵RA EQ Q RLK SQD LE¹⁰⁸-OH

The numbering in peptide #147 and #148 refers to the amino acids of the published membrane bound form of CD23. The double underlined amino acids (Q⁸¹ and L¹⁰²) correspond to the N-termini of the 37 kDa and 33 kDa fragments, respectively, isolated from the human B cell line (RPMI 8866 cells) cultured under serum free conditions without adding inhibitors for proteinases. The estimated cleavage sites with recombinant ADAM9-Fc are indicated by down-pointing arrows. The catalytically inactive ADAM9E/A-Fc protein neither cleaved the oxidized insulin B chain nor peptides #147 and #148.

Results

Recombinant ADAM9-Fc cleaves peptide #148 containing the 37 kDa cleavage site as well as peptide #147 containing the 33 kDa cleavage site near the cleavage sites obtained by N-terminal sequencing of the respective fragments isolated from B cell lines under shedding conditions (spontaneous release) under serum free conditions (see Kikutani, H. et al., 1986; Letellier et al., 1989; Sarfati et al., 1992). The cleavage of peptides #147 and #148 could not be blocked by serine-, cystein- and aspartyl-proteinase inhibitors. In contrast, the cleavage of both peptides could be blocked by EDTA, which blocks all metalloproteinase activities. In the case of s-CD23 fragments isolated earlier by others from serum free culture supernatants, one cannot exclude that proteinases other than the respective specific CD23 sheddase might have further degraded the initial cleavage product of the CD23 shedding enzyme. This is most likely the reason for the slightly different cleavage sites observed with the synthetic peptides.

The in vitro cleavage assays further demonstrate that soluble ADAM9 can cleave the CD23 peptides near the N-termini obtained under in vivo conditions and support further strong evidence for ADAM9 as a sheddase of CD23 in vivo.

These studies identify ADAM9 as a sheddase which cleaves CD23 on the membrane of CD23 positive cells. This is based on the following major new findings:

I: Cotransfection (COS cells, CHO cells) of ADAM9 and CD23 leads to the release of s-CD23 (mainly the 33 kDa form).

II: Shedding is not blocked by TIMP1, -2 and TIMP-3, known to inhibit all known MMPs, MT-MMPs as well as ADAM10 and ADAM17 (TACE).

III: Upregulation of ADAM9 expression in human B cells leads to an enhanced generation of s-CD23 which cannot be inhibited by TIMP-1, -2 and TIMP-3.

IV: Soluble ADAM9 both cleaves CD23 peptides containing the cleavage site of the 37 kDa fragment, as well as the 33 kDa fragment. The latter s-CD23 fragment (33 kDa) is a key regulatory molecule of IgE synthesis during allergic reactions.

### EXAMPLE 9

Materials and Methods

Cell Lines and Cell Culture

COS-7 cells were cultured in DMEM supplemented with 10% fetal calf serum, 2 mM glutamine, 2 mM pyruvate, and essential as well as non-essential amino acids (Gibco-BRL). The cells were split 1:3 the day before transfection. CHO AA8 cells (Clontech), containing the TC regulatable transactivator (Gossen and Bujard, 1991) were cultured like COS cells. The expression of the transactivator was repressed by adding 1 μg/ml TC (Sigma) to the medium. The human B cell line P493-6 (Schuhmacher et al., 1999) was cultured in RPMI1640 supplemented with 10% FCS and 1 μM β-estradiol (Sigma). To switch off c-myc expression 1 μg/ml TC was added to the culture medium.

cDNA Constructs

The full length cDNA of human CD23b was cloned into the expression vectors pcDNA3 (Invitrogen) and pBEHpac18 (Artelt et al., 1988). cDNAs for murine ADAM9, bovine ADAM10 (kindly provided by Prof. Fahrenholz) and murine ADAM17 (TACE) were cloned into pcDNA3 (Invitrogen). For tet-regulatable expression of ADAM9 in CHO M8 cells (Clontech), the full length cDNA was cloned into pBI (Clontech).

Transfection of Cells

COS-7 cells (5-6×10⁵ cells/10 cm dish) were cotransfected with human CD23 in pcDNA3 (5 mg) and either ADAM9, ADAM10-HA (kindly provided by Prof. Fahrenholz), and ADAM17 (all cloned in pcDNA3, 7.5 mg each) using the DEAE dextran method. Control transfections were performed with the respective expression vector alone. 16 h after transfection, the cells were trypsinized and seeded into new plates. 48 h after transfection, the cells were detached from the plates with cold PBS+2 mM CaCl₂ and analyzed for CD23 expression by FACS analyses using a FITC-labelled monoclonal antibody against human CD23 (Becton Dickinson).

CHO M8 cells (Clontech) grown in 6 cm dishes were transfected with 3.3 μg CD23b-pBEHpac18+6.6 μg pBI-ADAM9 using SuperFect essentially as recommended by the supplier (Qiagen). Clones were selected by adding 1 μg/ml TC+100 μg/ml G418+5 μg/ml puromycin to the culture medium. Single clones were picked and transferred to 24-well plates. All clones were tested by RT-PCR and Western blotting.

Influence of Metalloproteinase Inhibitors on CD23 Shedding

To inhibit matrix metalloproteinase activity, aliquots of the cells studied (transfected CHO AA8, P43-6) were washed three-times in serum free medium and further cultured either in the presence (1 μM TC) or absence of TC for 24-48 h, either in the presence of 2 μM batimastat (BB-94, British Biotechnology) or 20 nM-1 μM of either TIMP-1, -2, or TIMP-3. Control cells were further kept in the presence of 1 μM TC or absence of TC without inhibitors. Surface CD23 in the presence of either of the inhibitors was quantitated by FACS analyses using a FITC-labelled monoclonal antibody against human CD23 and compared to cells cultured in the absence of the respective inhibitor.

Quantification of s-CD23

100 μl aliquots of the culture supernatants from cells kept in the presence or absence of the respective inhibitor (see above) were used for the ELISA assay (BINDAZYME s-CD23 immunoassay kit, The Binding Site, Birmingham, UK or s-CD23 ELISA kit, Becton Dickinson). All culture supernatants were analyzed in duplicate in at least two different dilutions. The internal standard (s-CD23) was used for the calibration curve.

Peptide Cleavage Assays with Soluble ADAM9-Fc Fusion Protein

50 μM of each peptide studied (oxidized insulin B chain, peptide #147 and peptide #148) were incubated in the absence (peptide control) or presence of 1.35-2.70 ng of purified ADAM9-Fc in 20 μl assay buffer (100 mM Tris/HCl+100 mM NaCl+20 μM ZnCl₂+10 mM CaCl₂) at 37° C. for up to 16 h. The peptide cleavage products were purified using reverse phase tips as recommended by the supplier (ZIP-Tip, Millipore) and analyzed by mass spectrometry (Maldi-TOF).

Western Blot Analysis

a) ADAM9 expression: 2×10⁶ cells were washed serum-free with cold PBS. The cell pellets were directly lysed in 100 μl pre-boiled 2×Laemmli buffer for 5 min. Samples were centrifuged (5 min, 15000×g) and 15 μl aliquots were subjected to SDS PAGE (Laemmli, 1970). The separated proteins were transferred to PVDF membranes. Residual protein binding sites were blocked using PBS+0.25% Tween 20. ADAM9 was detected after incubation with rabbit anti-AD9Dis antibody, followed by an incubation with peroxidase-labelled goat anti-rabbit IgG (Dianova). Bound antibody was visualzed using the ECL detection system (Amersham Pharmacia Biotech).

b) TACE (ADAM17) expressed in COS cells was detected by Western blot analyses using an antibody kindly provided by Prof. Dr. C. P. Blobel, New York).

c) Bovine ADAM10 containing a HA-tag epitope at the carboxy terminus expressed in COS cells was detected using a specific HA-epitope specific monoclonal rat antibody (clone 3F10, Roche) as recommended by the supplier.

FACS Analysis of Surface CD23

10⁶ cells were washed once with PBS and were then incubated with 1 μg FITC-labelled mouse anti-human CD23 (mAb M-L233, Pharmigen) or the respective isotype control (mouse IgG1; clone 107.3, Pharmingen) in a final volume of 50 μl. After 45 min the cells were washed twice in 1 ml PBS. The cells were analyzed using a FACSort with CellQuest software (Becton Dickinson).

RT-PCR Analysis

RNA was isolated from 10⁶ cells using the High Pure RNA isolation kit (Roche). 5 μg of the RNA samples were reverse transcribed (RT) using M-MLV reverse transcriptase (Gibco BRL) using oligo-dT as a primer. 1 μl of RT mixtures was used for PCR amplification in a total volume of 25 μl containing 5 pmol of each primer and 0.75U of Taq polymerase (Qiagen). The primers used for amplification are summarized in Table 1.

TABLE 1
Primers used.

human ADAM9:

(SEQ ID NO:5)

| | |
|---|---|
| 5′-primer: | CCC CTA GGC CCT ATT CAA AA |

(SEQ ID NO:6)

| | |
|---|---|
| 3′-primer: | TGA ACT CCC TCC ACA TAG CC |

human CD23-3′:

(SEQ ID NO:7)

| | |
|---|---|
| 3′-Primer: | CTC TGT GTG GTG TCC CAG TG |

human CD23A:

(SEQ ID NO:8)

| | |
|---|---|
| 5′-primer: | GGG AGT GAG TGC TCC ATC AT |

human CD23B:

(SEQ ID NO:9)

| | |
|---|---|
| 5′-primer: | AAC AGG AAC TTG GAA CAA GCA |

human actin:

(SEQ ID NO:10)

| | |
|---|---|
| 5′-primer: | ACC AAC TGG GAC GAC ATG GA |

(SEQ ID NO:11)

| | |
|---|---|
| 3′-primer: | GCC ATC TCC TGC TCG AAG TC |

human c-myc:

(SEQ ID NO:12)

| | |
|---|---|
| 5′-primer: | TCA AGA GGC GAA CAC ACA AC |

(SEQ ID NO:13)

| | |
|---|---|
| 3′-primer: | TTT CCG CAA CAA GTC CTC TT |

human ADAM10:

(SEQ ID NO:14)

| | |
|---|---|
| 5′-primer: | TTC AGG AAG CTC TGG AGG AA |

(SEQ ID NO:15)

| | |
|---|---|
| 3′-primer: | TTC TCC TGG TGT GCA CTC TG |

human ADAM17:

(SEQ ID NO:16)

| | |
|---|---|
| 5′-primer: | TGC AGT GAC AGG AAC AGT CC |

(SEQ ID NO:17)

| | |
|---|---|
| 3′-primer: | GGA TGC ATT TCC CAT CCT TA |

human ADAM TS1:

(SEQ ID NO:18)

| | |
|---|---|
| 5′-primer: | CAA TGC CCT TCG ACC TAA AA |

(SEQ ID NO:19)

| | |
|---|---|
| 3′-primer: | CCA CTT CCT TTG CAC ACT CG |

human MT1-MMP (MMP14):

(SEQ ID NO:20)

| | |
|---|---|
| 5′-primer: | CTG GCT ACA GCA ATA TGG C |

(SEQ ID NO:21)

| | |
|---|---|
| 3′-primer: | ACC TTG GGG GTG TAA TTC TGG |

human MT2-MMP (MMP15)

(SEQ ID NO:22)

| | |
|---|---|
| 5′-primer: | GAA GAC GCG GAG GTC CAT GCC |

(SEQ ID NO:23)

| | |
|---|---|
| 3′-primer: | GAG GGC GTA GCG CTT CCG ACG |

human MT3-MMP (MMP16)

(SEQ ID NO:24)

| | |
|---|---|
| 5′-primer: | GGG GCT TGC CTC CTA GTA TC |

(SEQ ID NO:25)

| | |
|---|---|
| 3′-primer: | ATA GGT TTT CCC GAC GTC CT |

human MT4-MMP

(SEQ ID NO:26)

| | |
|---|---|
| 5′-primer: | TGG CCA GGA GTA CTG GAA AG |

(SEQ ID NO:27)

| | |
|---|---|
| 3′-primer: | CAG ACC TCG TAA CCG TCC TC |

human MT5-MMP:

(SEQ ID NO:28)

| | |
|---|---|
| 5′-primer: | CAT CCT GTG GTG TCC ATG AG |

(SEQ ID NO:29)

| | |
|---|---|
| 3′-primer: | CAG AAG CAG GGG TCC TGT AG |

human MT6-MMP:

(SEQ ID NO:30)

| | |
|---|---|
| 5′-primer: | CCA GAT ATC ACC CCT GAG GA |

(SEQ ID NO:31)

| | |
|---|---|
| 3′-primer: | CTT GGG GAT AGA AGG GTT CC |

human MMP1:

(SEQ ID NO:32)

| | |
|---|---|
| 5′-primer: | GAT GTG GAG TGC CTG ATG TG |

(SEQ ID NO:33)

| | |
|---|---|
| 3′-primer: | TGC TTG ACC CTC AGA GAC CT |

human MMP2:

(SEQ ID NO:34)

| | |
|---|---|
| 5′-primer: | CCA AGA CGG TCG TTT TGT CT |

(SEQ ID NO:35)

| | |
|---|---|
| 3′-primer: | GTT GAA GCG CCA GTA CCT GT |

human MMP7:

(SEQ ID NO:36)

| | |
|---|---|
| 5′-primer: | CAG ATG TGG AGT GCC AGA TG |

(SEQ ID NO:37)

| | |
|---|---|
| 3′-primer: | TGT CAG CAG TTC CCC ATA CA |

human MMP9:

(SEQ ID NO:38)

| | |
|---|---|
| 5′-primer: | AGT TCC CGG AGT GAG TTG AA |

(SEQ ID NO:39)

| | |
|---|---|
| 3′-primer: | CTC CAC TCC TCC CTT TCC TC |

### REFERENCES

Artelt, P., Morelle, C., Ausmeier, M., Fitzek, M., and Hauser, H. (1988) ,, Vectors for efficient expression in mammalian fibroblastoid and lymphoid cells via transfection or infection” Gene 68: 213-219

Bailey, S., Bolognese, B., Buckle, D. R., Faller, A., Jackson, S., Louis-Flamberg, P., McCord, M., Mayer, R., Marshall, L. A. and Smith, D. G. (1998) “Hydroxamate-based inhibitors of low affinity IgE receptor (CD23) processing” Bioorg. Med. Chem. Lett. 8:23-28.

Bailey, S., Bolognese, B., Buckle, D. R., Faller, A., Jackson, S., Louis-Flamberg, P., McCord, M., Mayer, R., Marshall, L. A., and Smith, D. G. (1998) “Selective inhibition of low affinity IgE receptor (CD23) processing” Bioorg. Med. Chem. Lett. 8:29-34.

Bettler, B., Maier, R., Ruegg, D., and Hofstetter, H. (1989) “Binding site for IgE on the low affinity Fcε receptor (Fcε-RII/CD23) is confined to the domain homologous with animal lectins” Proc. Natl. Acad. Sci. USA 86:7118-7122.

Black, R. A. and White, J. M. (1998) “ADAMs: focus on the protease domain” Curr. Opin. Cell Biol. 10:654-659.

Bode, W., Gomis-Ruth, F. X., and Stockler, W. (1993) “Astacins, serralysins, snake venom and matrix metalloproteinases exhibit identical zinc-binding environments (HEXXHXXGXXH and Met-turn) and topologies and should be grouped into a common family, the ‘metzincins’” FEBS Left. 331:134-140.

Christina, G., Barton, A., Bolognese, B., Buckle, D. R., Cook, R. M., Hansbury, M. J., Harper, G. P., Marshall, L. A., McCord, M. E., Moulder, K., Murdock, P. R., Seal, S. M., Spackman, V. M., Weston, B. J., and Mayer, R. J. (1997) “Inhibition of s-CD23 and immunoglobulin E release from human B cells by a metalloproteinase inhibitor, GI 129471” Eur. J. Immunol. 27:3228-3235.

Clark, E. A. (1990) “CD40: a cytokine receptor in search of a ligand” Tissue Antigens 35:33-36.

Defrance, T., Aubry, J. P., Rousset, F., Vanbervliet, B., Bonnefoy, J. -Y., Arai, N., Takebe, Y., Yokota, T., Lee, F., Arai, K., de Vries, J., and Banchereau, J. (1987) “Human recombinant interleukin-4 induces Fcε receptors (CD23) on normal B-lymphocytes” J. Exp. Med. 165:1459-1467.

Delespesse, G., Suter, U., Mossalayi, D., Bettler, B., Sarfati, M., Hofstetter, H., Kilcherr, E., Debre, P., and Dalloul, A. (1991) “Expression, structure and function of the CD23 antigen” Adv. Immunol. 49:149-191.

Gordon, J., Caims, J. A., Liu, Y. -J., Flores-Romeo, L., MacLennan, I. C., Jansen, K. U., and Bonnefoy, J. -Y. (1991) “Role of membrane and soluble CD23 in lymphocyte physiology” Monogr. Allergy 29:156-168.

Gossen, M. and Bujard, H. (1992) “Tight control of gene expression in mammalian cells by tetracycline-responsive promotors” Proc. Natl. Acad. Sci. USA 89:5547-5551.

Hooper, N. M., Karran, E. H., and Turner, A. J. (1997) “Membrane protein secretases” Biochem. J. 321:265-279

Kempkes, B., Spitkovsky, D., Jansen-Durr, P., Eilwart, J. W. Kremmer, E., Delecluse, H. J., et al. (1995) “B cell proliferation and induction of early GI-regulating proteins by Epstein-Barr virus mutants conditional for EBNA2” EMBO J. 14:88-96.

Kikutani, H., Inul, S., Sato, R., Barsumian, E. L., Owakim, H., Yamasaki, K., 'Kaisho, T., Uchibayashi, N., Hardy, R. R., Hirano, T., Tsunasawa, S., Sakiyama, F., Suemura and Kishimoto (1986) “Molecular structure of human lymphocyte receptor for immunoglobulin E” Cell 147:867-885.

Kleinau, S., Martinsson, P., Gustavsson, S. and Heyman, B. (1999) “Importance of CD23 for collagen-induced arthritis: delayed onset and reduced severity in CD23-deficient mice” J. Immunol. 162:4266-4270.

Laemmli, U. K. (1979) “Cleavage of structural proteins during the assembly of the head of baCTERIOPHAGE T4” Nature 227:680-685

Lebman, D. A. and Cofman, R. L. (1988) “Interleukin-4 causes isotype switching to IgE in T cell stimulated clonal B cell cultures” J. Exp. Med. 168,853-862.

Letellier, M., Nakajima, T., Pulido-Cejudo, T., Hofstetter, H., and Delespesse, G. 10 (1990) “Mechanism of formation of human IgE-binding factors (soluble CD23): III. Evidence for a receptor (Fc epsilon RII)-associated proteolytic activity” J. Exp. Med. 172:693-700.

Letellier, M., Sarfati, M., and Delespesse, G, (1989) “Mechanisms of formation of IgE binding factors (soluble CD23). Fc epsilon RII bearing B cells generate IgE binding factors of different molecular weights” Mol. Immunol. 26: 1105-1112.

Liu, Y. -J., Caims, J. A., Holder, M. J., Abbot, S. D., Jansen, K. -U., Bonnefoy, J. Y, Gordon, J., and MacLennan, I. C. M. (1991) “Recombinant 25-kDa CD23 and interleukin-Iα. promote the survival of germinal centre B cells: Evidence for birfurcation in the development of centrocytes rescued from apoptosis” Eur. J. Immunol. 21:1107-1114.

Marolewski, A. E., Buckle, D. R. Christie, G., Earnshaw, D. L., Flamberg, P. L. Marshall, L. -A., Smith, and Mayer, R. J. (1998) “CD23 (FcRII) release from cell membranes is mediated by a membrane-bound metalloproteinase” Biochem. J. 333:573-579.

Mayer, R. J., Bolognese, B. J., Al-Mahdi, N., Cook, R. M., Flamberg, P. L., Hansbury, M. J., Khandekar, S., Appelbaum, E., Faller, A. and Marshall, L. A. (2000) “Inhibition of CD23 processing correlates with inhibiton of IL-4 stimulated IgE production in human PBL and hu-PBL reconstituted SCID mice”. Clin. Exp. Allergy 30: 719-727.

Moss, M. L., Jin, S. -L. C., Milla, M. E., Burkhart, W., Cartner, H. L., Chen, W. -J., Clay, W. C., Didsbury, J. R., Hassler, D., Hoffman, C. R. et al. (1997) “Cloning a disintegrin metalloproteinase that processes precursor tumor-necrosis factor-α,” Nature 385:733-736.

Munoz, O., Brignone, C., Grenier-Brosette, N., Bonnefoy, J. Y., and Cousin, J. L. (1998) “Binding of anti-CD23 monoclonal antibody to the leucine zipper motif of FcεRII/CD23 on B cell membrane promotes its proteolytic cleavage. Evidence for an effect on the oligomer/monomer equilibrium” J. Biol. Chem. 273:31795-31800.

Punnonen, J., Aversa, G., Cocks, B. G., McKenzie, A. N., Menon, S., Zurawski, G., de Waal Malefyt, R., and de Vries, J. E. (1993) “Interleukin-13 induces interleukin-4 independent IgG4 and IgE synthesis and CD23 expression in human B cells” Proc. Natl. Acad. Sci. USA 90:3730-3734.

Roghani M, Becherer J D, Moss M L, Atherton R E, Erdjument-Bromage H, Arribas J, Blackburn R K, Weskamp G, Tempst P, Blobel C P (1999). “Metalloprotease-disintegrin MDC9: intracellular maturation and catalytic activity” J. Biol. Chem. 274:3531-3540

Schlöndorff, J. and Blobel, C. P. (1999) “Metalloprotease-disintegrins: modular proteins capable of promoting cell-cell interactions and triggering signals by protein-ectodomain shedding” J. Cell Sci. 112:3603-3617.

Schuhmacher, M., Staege, M. S., Pajic, A., Polack, A., Weidle, U. H., Bomkamm, G. W., Eick, D., and Kohlhuber, F. (1999) “Control of cell growth by c-myc in the absence of cell division” Curr. Biol. 9:1255-1258.

Weskamp, G., Krätzschmar, J. R., Reid, M., and Blobel, C. P. (1996) “MDC9, a widely expressed cellular disintegrin containing cytoplasmic SH3 ligand domains” J. Cell Biol. 132:717-726.

Yokota, A., Kikutani, H., Tanaka, T., Sato, R., Barsumian, E. L., Suemura, M., and Kishimoto, T. (1988) “Two species of human Fcε receptor II (Fcε-RII/CD23): Tissue-specific and IL-4-specific regulation of gene expression” Cell 55:611-618.

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

I claim:

1. A composition for treatment or prophylaxis of allergy in which overproduction of soluble CD23 (s-CD23) is implicated, comprising an inhibitor for human s-CD23 formation, wherein the inhibitor selectively decreases or blocks the activity of the metalloprotease ADAM9, wherein ADAM9 mediates the shedding of s-CD23 in human B cell lines.

2. The composition according to claim 1, wherein the inhibitor is a monoclonal or polyclonal antibody which is selectively directed against the metalloprotease ADAM9.

3. The composition according to claim 1, wherein the inhibitor is a synthetic inhibitor selected from the group consisting of hydroxamic acid based and barbituric acid based inhibitors.

4. A composition for treatment of allergy in which overproduction of s-CD23 is implicated, comprising an inhibitor for human soluble CD23 formation, wherein the inhibitor selectively decreases or blocks the activity of the metalloprotease ADAM9.

5. The composition according to claim 4, wherein the inhibitor is a monoclonal or polyclonal antibody which is selectively directed against the metalloprotease ADAM9.

6. The composition according to claim 4, wherein the inhibitor is a synthetic inhibitor selected from the group consisting of hydroxamic acid based and barbituric acid based inhibitors.

7. A composition for inhibiting shedding of s-CD23 in human B cell lines comprising an inhibitor for human s-CD23 formation, wherein the inhibitor selectively decreases or blocks the activity of the metalloprotease ADAM9.

8. The composition according to claim 7, wherein the inhibitor is a monoclonal or polyclonal antibody which is selectively directed against the metalloprotease ADAM9.

9. The composition according to claim 7, wherein the inhibitor is a synthetic inhibitor selected from the group consisting of hydroxamic acid based and barbituric acid based inhibitors.

10. An antibody which selectively binds to the metalloprotease ADAM9.

11. The antibody according to claim 10, wherein the antibody is monoclonal.

12. The antibody according to claim 10, wherein the antibody is humanized.

13. A composition comprising the antibody according to claim 10 and a pharmaceutically acceptable excipient.

14. A method of treating allergy comprising administering a therapeutically effective amount of the composition of claims 1, 4 or 7 to a patient in need thereof.

15. The method according to claim 14, wherein the inhibitor is a monoclonal or polyclonal antibody which is selectively directed against the metalloprotease ADAM9.

16. The method according to claim 14, wherein the inhibitor is a synthetic inhibitor selected from the group consisting of hydroxamic acid based and barbituric acid based inhibitors.
